(19) United States
(12) Patent Application Publication
(10) Pub. No.: US 2004/0030404 A1
(43) Pub. Date: Feb. 12, 2004

(54) Method for cultivating a cartilage replacement and a biomatrix produced according to this method

(76) Inventors: Michaela Noll, Stuttgart (DE); Markus Schandar, Oberstenfeld (DE); Thomas Graeve, Stuttgart (DE)

Correspondence Address: Dickstein Sharpiro Morin & Oshinsky, 1177 Avenue of The Americas, 41st Floor, New York, NY, 10036-2714, US

(21) Appl. No.: 10/296,966

(22) PCT Filed: May 29, 2001
(86) PCT No.: PCT/EP01/06073

(30) Foreign Application Priority Data
May 31, 2000 (DE) 10026789.0

(51) Int. Cl. A61F 2/02

(52) U.S. Cl. 623/23.63; 623/23.72; 623/13.17

(57) ABSTRACT

The present invention relates to methods for cultivating cartilage cells, methods for redifferentiating dedifferentiated cartilage cells, as well as cartilage transplants.

### FIELD OF THE INVENTION

The invention relates to methods for cultivating differentiated and dedifferentiated cartilage cells, a cartilage replacement, a biomatrix, a cell-matrix-cartilage system as well as methods for their production.

### BACKGROUND OF THE INVENTION

Joint diseases are a widespread disorder associated with a large number of problems for the persons afflicted. Bone and cartilage defects, for example, play a central role in the pathogenesis of arthrosis, but also in post-traumatic conditions and loosened endoprostheses. Such defects can be treated by using autologous or homologous bone or cartilage or appropriate replacement materials. While on the one hand the supply of autologous materials is not unlimited, infectological aspects must be taken into account for homologous transplants.

The main problem of degenerative or traumatic joint diseases is that the damaged joint cartilage shows only little ability for regeneration. A known treatment of such bone defects is the autologous chondrocyte transplantation (ACT) as a biological therapy. With this therapy, the formation of new hyaline cartilage in a joint cartilage defect could for the first time be demonstrated.

The principle of this method is based on injecting cartilage cells of the patient, which were cultivated in vitro, under a periosteum flap following debridement of the degenerated cartilage (Peterson method). This method is technically difficult and poses methodic problems; it is, for example, not guaranteed that the cartilage cells will remain in the defect. It is also unclear whether the cultivated cartilage cells are at the time of transplantation able to form the matrix necessary for the permanent filling of the defect. The transplanted cells are mostly in a dedifferentiated state and have morphological and physiological commonalities with fibroblasts.

From DE 197 21 661 A1, the use of innovative materials for replacing bone or cartilage that are characterized by a specific structure of actually known materials is known. This relates to a bone or cartilage implant based on a three-dimensional grid that consists essentially of a large number of regularly arranged bars of a partially or completely bioabsorbable material. The bars form a geometric, three-dimensional structure, whereby this structure is adapted with respect to elasticity and strength to the tissue it is meant to replace in the patient's body. The bars may consist of poly-D-lactides, poly-L-lactides, poly-DL-lactides, hydroxy apatites, calcium phosphates, or mixtures of these substances that essentially contain calcium phosphates or hydroxy apatites, collagen, agar, or gelatin.

WO 99/08728 discloses an osteoinductive or chondroinductive factor mixture embedded in nanospheres. Also disclosed is a system comprising a biodegradable matrix containing, for example, collagen type I or type II and, imbedded in it, the factors enveloped by the nanospheres. The nanospheres are realized as polymer particles.

WO 95/33821 discloses the production of three-dimensional structures from collagen, whereby the interstitial spaces are bridged, for example, by fibroblasts or chondrocytes, and a cultivation of this network is performed in a culture medium. This document describes the cultivation of said cells on this three-dimensional network, as well as the use of this artificial tissue as a cartilage replacement.

WO 98/17791 describes the extraction and use of precursor chondrocytes that can be methodically multiplied and used as a therapeutic cartilage tissue. This document also describes the cultivation of chondrocytes on a three-dimensional network, whereby the interstitial spaces are bridged with chondrocytes.

WO 99/00152 discloses a method for producing a bioartificial transplant, whereby all antigen-reactive cells are removed by enzymatic or chemical treatment from an allogenic or xenogenic tissue, and the obtained cell-free, non-denatured material is colonized with desired, autologous cells, resulting in an immediately usable transplant.

### BRIEF SUMMARY OF THE INVENTION

The disadvantage of the known methods is that transplanted or cultivated cartilage cells do not resume their original synthesis action.

The reason for this is that the cartilage cells—chondrocytes—embedded in the intracellular substance of the cartilage dedifferentiate under in vitro culture conditions during the course of the cultivation period. While primary cartilage cells (P0 culture) still show cartilage-cell-specific synthesis actions after being extracted from cartilage tissue, as is shown by the formation of collagen type II, type IX, and type XI, cultivated cells lose these typical characteristics during further passages (P1-PX) in vitro.

In contrast to other cells, cartilage cells also are hard to cultivate. Cultivated cartilage cells are in a dedifferentiated state, and morphologically and physiologically resemble fibroblasts. Especially larger defects require the multiplication of the autologous chondrocytes by cultivation and subjecting to passages for the production of adequate amounts of transplantation material from small samples.

Up to now, the dedifferentiation of the cartilage cells could only be prevented by adding additional growth stimulants, which makes the in vivo use of these cultures more difficult since the growth stimulants may adversely interact with the immune system of the recipient's organism. The known methods furthermore do not ensure that the cells maintain their natural or almost natural metabolism even after several passages during cultivation.

The known three-dimensional structures for cultivating cartilage cells also cannot be specifically adapted to the respective cartilage defect. It is furthermore not ensured that the transplanted cartilage cells remain permanently and differentiated at the site of the defect and in this way enable regeneration of the cartilage.

Indeed, there is so far no transplantation method that ensures that transplanted cartilage cells permanently remain at the site of the defect thus ensuring the associated regeneration of the cartilage.

The technical objective underlying the present invention is therefore to provide cartilage transplants, as well as methods and means for their production, that permit an improved treatment of cartilage diseases, in particular an improved healing or regeneration of the treated cartilage defect.

The invention realizes the underlying objective in particular by providing a method for the redifferentiation and/or multiplication of dedifferentiated cartilage cells, whereby the dedifferentiated cartilage cells are cultivated in a three-dimensional, gel-like biomatrix, where they redifferentiate, and resume their cell-specific metabolic actions. The biomatrix according to the invention contains a collagen network, newly constituted from a preferably fresh collagen solution, with a concentration of at least 1.5 mg collagen/ml of biomatrix, preferably 1.5 to 4 mg/ml of biomatrix. This collagen network is obtained from a preferably cell-free, acidic collagen solution, preferably with a pH value of 0. 1 to 6.9, preferably 2.0 to 5.0, especially 3.0 to 4.5, in particular 3.2 to 4.2, and especially preferred 3.8, said collagen solution having been prepared and stored at 2 to 10° C., preferably at 4° C. In order to prepare a cell-free biomatrix, a solution of medium, in particular a conventional cell culture medium, buffer, for example HEPES buffer, and serum, in particular human autologous serum, is added at 2 to 10° C., preferably at 4° C., to this collagen solution, which is then gelled by increasing the temperature to, for example, room temperature or 37° C. In order to prepare a cell-containing biomatrix, preferably precultivated cells, for example cartilage cells or precursor cartilage cells, are added to the solution of medium, buffer, and serum, to which is then added, at 2 to 10° C., preferably at 4° C., the collagen solution that also has been brought to a temperature of 2 to 10° C., preferably 4° C. The cells embedded into the biomatrix in this way then can be cultivated and, if necessary, be removed again. This is followed by gelling, for example at room temperature or at 37° C., and the biomatrix is coated with cell culture medium. The method according to the invention advantageously makes it possible to perform an intermediate cultivation of the cells in a biomatrix according to the invention, whereby the chondrocytes, for example of the P2 culture, are stimulated in the three-dimensional biomatrix to a resynthesis of cell-specific matrix proteins, especially collagen II, whereas no collage formation can be demonstrated in known cultures. It is advantageous that the cells can be removed again from the biomatrix and cultivated. The redifferentiation of dedifferentiated cartilage cells according to the invention makes it possible to cultivate and/or multiply chondrocytes over a longer period without the cells losing the specific synthesis actions necessary for building cartilage. This means that even with a small starting amount of cartilage tissue, sufficient cell material for producing cartilage transplants can be made available in an advantageous manner.

In connection with the present invention, the term “cultivation of cells” means a preferably in vitro maintenance of the life functions of cells in a suitable environment, for example by adding and removing metabolic educts and products, in particular also for the multiplication of the cells.

In connection with the present invention, the terms “cartilage cells” or “chondrocytes” stand for naturally occurring or genetically engineered cartilage cells or their precursors that may be of animal or human origin.

A further embodiment of the invention provides for the cultivation of differentiated cartilage cells, especially with preservation of their differentiation, in a three-dimensional biomatrix as characterized above. Advantageously, the metabolic actions of the primary culture are preserved without a dedifferentiation of the differentiated cartilage cells taking place. This means that the invention also provides a cultivation method for chondrocytes that comprises a suppression of the redifferentiation.

Another advantageous embodiment of the invention provides that cartilage cells to be tested with respect to their function, morphology and/or differentiation status are introduced into said three-dimensional biomatrix, are cultivated, and are simultaneously and/or subsequently tested. It is hereby advantageous that in this manner, for example, the metabolism of the cartilage cells can be tested in vitro before the cartilage cells are implanted in vivo. The test may for example be the measurement of metabolic actions as well as a morphological or functional test.

The invention therefore also relates to a screening and diagnosis method, whereby cartilage cells are cultivated according to the previously described methods and simultaneously and/or subsequently can be tested, for example for physiological, morphological and/or molecular-biological parameters. In particular, degenerative or traumatic joint diseases or the absence thereof can hereby be determined. Within the context of these methods, it is also possible to study the effects of potential medications and/or pathogens, antigens, etc. on the cultivated cells, for example in drug screening processes. In a preferred embodiment, the cells are cultivated in the presence and absence of the agent to be studied, and the observed effects are compared with each other.

In another advantageous embodiment of the invention, the cartilage cells are removed from the three-dimensional biomatrix, after having been cultivated in the same, for example with a collagenase treatment and subsequent concentrating, and undergo further cultivation in a standard, two-dimensional or three-dimensional cell culture. In this way, the advantages of the two-dimensional and three-dimensional cultivation can be combined in an advantageous manner.

In an especially advantageous embodiment of the invention, the cartilage cells are introduced in a three-dimensional biomatrix according to the invention and cultivated in the same in such a way that subsequently a transplantable cartilage replacement, also called a cartilage transplant, can be obtained. In a preferred embodiment, this cartilage replacement can be a joint cartilage replacement. This means that the biomatrix already can be advantageously adapted to the shape of the cartilage defect during the cultivation phase.

In an advantageous embodiment, the invention also relates to a previously mentioned method for producing a cartilage replacement, especially joint cartilage replacement, whereby the cartilage cells are removed from the three-dimensional biomatrix after having been cultivated in the same, preferably with a collagenase treatment and by centrifugation, and then undergo continued cultivation in—preferably—higher cell density in a standard two- or three-dimensional cell culture, whereby a transplantable cartilage replacement, in particular joint cartilage replacement, can be obtained.

The invention also relates to a cartilage replacement, in particular joint cartilage replacement, produced in accordance with the method according to the invention and a potentially subsequent or/and preceding cultivation method of a standard type.

The invention also relates to a preferably gel-like biomatrix, in which the previously mentioned cultivation processes can be performed, i.e. a biomatrix both without cartilage cells as well as a biomatrix with cartilage cells. In the latter case, the combination of biomatrix and differentiated or redifferentiated cartilage cells cultivated therein is also called a cell-matrix-cartilage system or biograft. This biograft can be used directly for treating cartilage diseases or defects.

According to the invention, the term “biomatrix” stands for a gel structure that contains collagen, cell culture medium, serum, and buffer, in particular HEPES buffer. The collagen solution used for preparing the biomatrix is a solution with a high content of non-denatured, native collagen in an acidic, aqueous medium, in particular with a pH value of 0.1 to 6.9, preferably 2.0 to 5.0, especially 3.0 to 4.5, in particular 3.2 to 4.2, and especially preferred 3.8, for example in acetic acid, preferably in 0.1% acetic acid solution. A high content of non-denatured collagen means a total collagen content in the solution of ≧50%, in particular ≧60%, ≧70%, ≧80%, ≧90%, or ≧95%, in particular ≧99%. In a preferred embodiment, no lyophilized collagen is used for this. The collagen content of the solution is preferably between 3 mg of collagen per ml of solution to 8 mg of collagen per ml of solution, preferably 6 mg of collagen per ml solution. It is advantageous that in the preferred embodiment collagen is used, which, after extraction, for example from rat tails, was incubated in 0.1% acetic acid for 3 to 14 days at 4° C. with stirring, and whereby undissolved collagen parts were centrifuged off. The preferred cell culture medium is DMEM (Dulbecco's Modified Eagle Medium). However, it is possible to use any desired cell culture medium that permits the cultivation of cartilage cells. In the preferred embodiment, it is advantageous that the serum is autologous human serum, and the buffer is, for example, HEPES buffer. In the preferred embodiment, a 3M concentration of HEPES is adjusted in this solution. The pH value of the solution of cell culture medium, buffer, and serum in the preferred embodiment is 7.5 to 8.5, for example 7.6 to 8.2, in particular 7.8. Naturally, the biomatrix may contain further factors, for example growth factors, adhesives, antibiotics, selection agents, etc.

The invention therefore also relates to methods for preparing a biomatrix that contains cells, whereby, in a first step, fresh collagen, for example from rat tails, is prepared by collecting collagen fibers extracted from collagen-containing tissue in buffer solution, superficially disinfecting them in alcohol, and then washing them in buffer solution and transferring them into an acidic solution with a pH value of 0.1 to 6.9, preferably 2.0 to 5.0, especially preferably 3.0 to 4.0, in particular 3.3, for example a 0.1% acetic acid solution. In a further step, the collagen in the solution is stirred at 2 to 10° C., in particular at 4° C., for several days, for example for 3 to 14 days, the undissolved collagen parts are centrifuged off, and a finished collagen solution with a collagen content of 3 mg/ml to 8 mg/ml is stored at 2 to 10° C., for example at 4° C. It is naturally also possible to temporarily store the solution in a frozen state, for example at −10° C. to —80° C., in particular at −20° C. In a third step, this collagen solution is mixed with a solution with a pH value from 7.5 to 8.5, preferably 7.6 to 8.2, in particular 7.8, containing double-concentrated cell culture medium, serum, and buffer in a ratio of preferably 1:1, resulting in a biomatrix with a pH value of 7.0 to 7.8, preferably 7.4. To prepare the biograft, the solution of double-concentrated cell culture medium, serum, and buffer is mixed with precultivated and centrifuged-off cartilage cells, whereby preferably 2×10⁴ to 2×10⁷ per ml, preferably 2×10⁶ cells, are used. This solution with a pH value of 7.5 to 8.5, preferably 7.6 to 8.2, in particular 7.8, is then mixed in a ratio of 1:1 with the previously mentioned collagen solution at 2 to 10° C., in particular at 4° C. The gel solution is then pipetted into culture containers and is coated, after gelling at 37° C., with medium. The biograft is then cultivated for 3 to 8 days, after which time it is available for transplantations.

The biograft is brought into the operating room in culture dishes and then can be adapted by the clinician to the defect in size, thickness, and shape by mechanical modification, for example with a knife. The biograft then is fixed, for example with a tissue adhesive, especially fibrin adhesive, in the defect. After approximately 5 minutes, the biograft is firmly anchored in the defect. The surgery also can be performed arthroscopically since the biograft is flexible.

Another use of the biograft is a combination of the biomatrix according to the invention with bone. The biograft is hereby, for example, applied to autologous bone cylinders from the iliac crest, using, for example fibrin adhesive. The invention enables especially the therapy of osteochondrosis dissecans and defects of the femur head in cerebral palsy-induced hip luxation as well as the remediation of traumatic and degenerative lesions of the knee joint. According to the invention, early and causal therapy is able to delay a progression of the traumatically or degeneratively induced joint diseases. The invention provides a unique and new therapy for treating arthrosis in younger people, which significantly delays the endoprosthetic joint replacement and, in individual cases, even makes it unnecessary. Joint destruction due to inflammatory causes also can be treated with the method according to the invention.

The development of the gel-like biomatrix according to the invention, into which autologous cells can be embedded in an exactly defined manner, for the first time enables the transplantation of a defined amount of cartilage cells in a mechanically shapeable and stressable matrix. The cell-matrix-cartilage system according to the invention, constructed of the biomatrix according to the invention and of cartilage cells, may be prepared in any desired size and thickness and can be exactly adapted to the defect through mechanical processing. The biograft according to the invention also offers the advantage that, in contrast to ACT, no additional periosteum removal from the patient's tibia is necessary. By cultivating the cell-matrix-cartilage system according to the invention in vitro, it is possible to study cartilage cells prior to transplantation with respect to their matrix production. This is an important condition for the production of cartilage transplants, their testing for functionality, and for quality assurance. It could be shown, for example, that the new synthesis of typical cartilage proteins, such as, for example, collagen type II, which provide the primary stability of the transplant, are formed by the autologous cartilage cells in vitro in the matrix. According to the invention, studies of the knee joint of minipigs were able to show that compared to the empty defect when transplanting empty matrix without cartilage cells or cartilage cell suspensions according to Peterson, the cell-matrix-cartilage systems heal extremely well into the joint cartilage, completely fill the defect, and are also stabile with respect to pressure.

Finally, the invention also relates to a biomatrix to be used in one of the previously mentioned methods.

The invention furthermore also relates to methods for treating cartilage diseases or defects, especially degenerative, inflammatory and/or traumatic joint diseases, whereby a cartilage replacement prepared according to the present invention, a biograft, and/or cartilage cells cultivated according to the invention is/are implanted into the diseased or damaged tissue or cartilage or bone areas or is/are used to replace the diseased or defective areas.

### DETAILED DESCRIPTION OF THE INVENTION

The invention is described in more detail using examples.

### EXAMPLE 1

### Collagen Preparation

Collagen-containing tissue, for example tendons from rat tails, is used to prepare a collagen solution. All work is performed under sterile conditions with sterile materials. After storage at −20° C., the rat tails are superficially disinfected with 70% alcohol for 5 minutes. The rat tails are skinned, and the individual collagen fibers are extracted. If using other starting tissues, any possibly present cells may be carefully removed with a mechanical, enzymatic, or chemical treatment. The collagen fibers are collected in phosphate-buffered saline (PBS) (pH 7.2), superficially disinfected in 70% alcohol for 10 minutes, and then washed thoroughly with PBS. The weight of the fibers is determined, and the fibers are transferred into a 0.1% acetic acid solution (approximately 8 to 12 mg/ml). This preparation is stirred for a period of 3 to 14 days at 4° C., and any undissolved collagen parts are then removed using centrifugation (1,000 rpm, 1 hour, 8° C.). In a preferred embodiment, the finished collagen solution has a collagen content of 3 mg/ml to 8 mg/ml. The collagen therefore is present as a starting material, with a high content, as previously defined, in solution, and not in fiber, network or matrix form. The obtained collagen solution preferably is cell-free and is prepared from fresh, non-lyophilized collagen.

### EXAMPLE 2

### Preparation of the Biomatrix or Biograft

All work is performed under sterile conditions with sterile materials.

| | |
|---|---|
| Collagen solution: | min. 3 mg/ml in acetic acid |
| Buffer solution: | 77.5 ml double-concentrated medium |
| | (e.g. DMEM) |
| | 20 ml serum |
| | 2.5 ml HEPES solution (3 M, ph 7.8) |

Both solutions are stored at 4° C.

The biomatrix consists of a collagen solution in 0.1% acetic acid (collagen from rat tails with a collagen content of 3 mg/ml to 8 mg/ml, preferably 6 mg/ml) and a buffer solution of double-concentrated medium, serum, and HEPES solution. Shortly after mixing these two components in a ratio of preferably 1:1, a three-dimensional, newly constituted collagen structure gels at temperatures above 4° C., for example at room temperature.

To prepare the biograft, i.e. a cell-containing biomatrix, 2×10⁴ to 2×10⁷ cartilage cells/ml, preferably 2×10⁶, which were precultivated and centrifuged off (1,000 rpm, 10 min., room temperature) in the usual manner, are placed in the buffer solution, are suspended, and mixed with equal parts of the collagen solution at 4° C. This gel solution is pipetted into culture dishes and, after gelling at 37° C. into a newly constituted collagen structure with embedded cartilage cells, is coated with medium. The material is available for transplantation after a culture period of 3 to 8 days.

If desired, the cells can again be released and obtained from the biomatrix with a collagenase treatment and subsequent centrifuging. The obtained, differentiated cells can be the starting point for further cultivations.

### EXAMPLE 3

### Transplantations in the Open Joint and Arthroscopic Application

Both during transplantation in the open joint and during arthroscopic application, the cell-matrix cartilage is first adapted mechanically in its size and shape to the cartilage defect. The transplant is then introduced into the defect and is fixed there using fibrin adhesive. Since the described cell-matrix-cartilage system is a flexible yet dimensionally stable material, it is possible to quasi roll this biograft into the cylindrical arthroscopy instruments and introduce it into the defect in this way. Because of their dimensional stability, the transplants resume their original shape at the target sire and then can be fixed in the defect with fibrin adhesive.

### EXAMPLE 4

### Results of PCR Tests

The following table reflects the results of PCR tests. The objective of these tests was to demonstrate that the cultivation according to the invention in a biomatrix according to the invention prepared according to Example 1 and 2 results in a redifferentiation of chondrocytes. The table shows that, starting from a P0 culture, the ability of the chondrocytes to produce collagen type II is lost during the course of the subsequent, conventionally performed passages P1 and P2. The cells dedifferentiate during the course of this passage. A P2 passage in a biomatrix according to the invention in contrast leads to the redifferentiation of dedifferentiated chondrocytes, which is confirmed by the recovered ability of being able to produce collagen type II.

TABLE
Results from PCR tests:
RNA was isolated from the chondrocytes of the individual
cultures and was transcribed into cDNA.

| | | | | P2-collagen |
|---|---|---|---|---|
| | P0-2D | P1-2D | P2-2D | 2 × 10⁶/ml |
| | 3 weeks | 2 weeks | 3 weeks | 3 weeks |
| ∃2 Microglobulin | + | + | + | + |
| Collagen type I | + | + | + | + |
| Collagen type II | + | − | − | + |
| Collagen type XI | + | + | + | + |
| Bmp 2 | + | + | + | + |
| Bmp 4 | + | + | + | + |
| Bmp 7 | − | − | − | − |

The expression (transcription) of chondrocyte-specific genes was tested by PCR amplification with suitable primers and subsequent gel electrophoresis:

∃2 Microglobulin is a constitutively expressed gene and functions as a positive control;

Collagen type II and XI are cartilage-specific collagen types;

Bone morphogenetic proteins (Bmp) 2, 4, and 7 play a role in the differentiation of the cartilage tissue.

1. Method for the redifferentiation and/or multiplication of dedifferentiated cartilage cells, wherein dedifferentiated cartilage cells are cultivated imbedded in a three-dimensional, gel-like biomatrix containing at least 1.5 mg/ml of collagen in buffered, serum-containing cell culture medium.

2. Method for the multiplication of differentiated cartilage cells, wherein the differentiated cartilage cells are cultivated, while preserving their differentiation, imbedded in a three-dimensional, gel-like biomatrix containing at least 1.5 mg/ml of collagen in buffered, serum-containing cell culture medium.

3. Method for testing the function, morphology and/or differentiation status of cartilage cells, wherein the cartilage cells to be tested are introduced into a three-dimensional, gel-like biomatrix containing at least 1.5 mg/ml of collagen in buffered, serum-containing cell culture medium, are cultivated in it, and are tested simultaneously or subsequently.

4. Method according to one of the previous claims, wherein the cartilage cells, after cultivation in the three-dimensional, gel-like biomatrix, are removed from the same and undergo further cultivation in a standard, preferably two-dimensional cell culture.

5. Method for producing a cartilage replacement, wherein cartilage cells are introduced into a three-dimensional, gel-like biomatrix containing at least 1.5 mg/ml of collagen in buffered, serum-containing cell culture medium and are cultivated therein in such a way that a transplantable cartilage replacement is obtained.

6. Method according to claim 5, wherein the cartilage cells, after cultivation in the three-dimensional, gel-like biomatrix, are removed from the same and undergo further cultivation in a higher cell density so that a transplantable cartilage replacement is obtained.

7. Method according to one of claims 1 to 6, wherein the biomatrix contains 1.5 mg/ml to 4 mg/ml collagen.

8. Method according to one of claims 1 to 7, wherein the biomatrix was prepared in that collagen fibers, extracted from a tissue, are stirred in acidic solution for 3 to 14 days at 2 to 10° C., preferably at 4° C., undissolved collagen parts are centrifuged off, and the resulting finished collagen solution with a collagen content of 3 mg/ml to 8 mg/ml is mixed with a solution containing cell culture medium, serum, and buffer at 2 to 10° C., preferably at 4° C., and is then gelled at a higher temperature, room temperature up to 37° C.

9. Cartilage replacement, prepared according to a method according to one of claims 5 to 8.

10. Method for preparing a biomatrix for the cultivation of cells, in particular cartilage cells, comprising the extraction of collagen-containing tissue, the transfer of the collagen-containing tissue into acidic solution, the incubation of the collagen tissue transferred into acidic solution at 2 to 10° C., in particular at 4° C., the centrifuging off of undissolved collagen parts, the mixing of the obtained collagen solution at 2 to 10° C., preferably at 4° C., with a solution containing cell culture medium, serum, and buffer, and the gelling of the mixed solution by increasing the temperature.

11. Method according to claim 10, wherein the acidic solution is acetic acid solution, in particular 0.1% acetic acid solution.

12. Method according to one of claims 10 or 11, wherein the solution containing cell culture medium, serum, and buffer is mixed in a ratio of 1:1 with the collagen-containing solution.

13. Method according to one of claims 10 to 12, wherein cartilage cells are added to the solution containing cell culture medium, serum, and buffer prior to the mixing with the collagen solution.

14. Biomatrix prepared according to one of the methods of claims 10 to 13.
